(19) United States
(12) Patent Application Publication
(10) Pub. No.: US 2010/0286642 A1
(43) Pub. Date: Nov. 11, 2010

(54) WATER-STABLE, OIL-MODIFIED, NONREACTIVE ALKYD RESIN CONSTRUCTION ADHESIVES, AND USE THEREOF

(76) Inventors: William Maxwell Allen, JR., Liberty Twshp., OH (US); Isao Noda, Fairfield, OH (US); Wolfgang Edgar Huhn, Chieti (IT)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/463,659

(22) Filed: May 11, 2009

(51) Int. Cl. A61F 13/15; C08K 5/09; B32B 7/12

(52) U.S. Cl. 604/366; 523/111; 156/327

(57) ABSTRACT

Disclosed herein is a new use for water-stable, oil-modified, nonreactive alkyd resins. It has now been found that such resins can be used as the predominant component of a construction adhesive employed in the manufacture of disposable absorbent articles, for example. As such, the construction adhesive need not employ plasticizers, tackifiers, and conventional polymers required by hot-melt adhesives typically used as construction adhesives. Furthermore, in the manufacture of disposable absorbent articles, the construction adhesive containing a water-stable, oil-modified, nonreactive, alkyd resin does not need to be processed or applied at the high temperatures required of construction adhesives containing reactive alkyds.

### FIELD OF THE INVENTION

The disclosure generally relates to alkyd resin-containing construction adhesives and use thereof. More specifically, the disclosure relates to construction adhesives predominantly containing water-stable, oil-modified, nonreactive alkyd resins, absorbent articles containing these adhesives, and a method of making the articles.

### BACKGROUND OF THE INVENTION

Adhesives are commonly used in the manufacture of disposable absorbent articles such as, for example, infant and adult incontinence articles and diapers. These articles are generally manufactured by combining several components and, more specifically, by employing adhesives to form an adhesive bond between different components of the article, thereby joining those components together. The components generally include a liquid-permeable topsheet, a liquid-impermeable backsheet attached to the topsheet, and an absorbent core positioned between the topsheet and the backsheet. Thus, for example, adhesives have been used to bond the topsheet and the backsheet together. Adhesives also have been used to bond discrete components, such as fasteners and leg elastics or cuffs, to each other or to the article. These adhesives are often referred to as construction adhesives because they are used to help construct the absorbent article from its individual components. In many instances, a hot-melt adhesive is used as a construction adhesive.

As used herein, “hot-melt adhesive” refers to a thermoplastic polymer composition that is heated to obtain a liquid of flowable viscosity, and after application to an adherend, cooled to obtain a solid. An adhesive bond is formed when the adhesive solidifies upon cooling to a temperature below its melt temperature or below its solidification transition temperature. Hot-melt adhesives used as construction adhesives in the manufacture of disposable absorbent articles typically include several components. These components include one or more polymers to provide cohesive strength, such as ethylene-vinyl acetate, copolymers, polypropylene, phenoxy resins, styrene-butadiene copolymers, ethylene-ethyl acrylate copolymers, low density polypropylenes, polyesters, polyamides, and polyurethanes. These polymers make up a significant part of the hot-melt adhesive composition. The composition also includes components such as, for example, a resin or analogous material (sometimes called a tackifier) to provide adhesive strength. Examples of such materials include hydrocarbons distilled from petroleum distillates, rosins and/or rosin esters, and terpenes derived, for example, from wood or citrus. The composition also typically includes waxes, plasticizers or other materials to modify viscosity. Examples of such materials include mineral oil, polybutene, paraffin oils, ester oils, and the like. Still further, the composition can optionally include additives, such as antioxidants or other stabilizers. A typical hot-melt adhesive composition might contain from about 15 to about 35 weight percent (wt. %) cohesive strength polymer(s); from about 50 to about 65 wt. % resin or other tackifier(s); from more than zero to about 30 wt. % plasticizer or other viscosity modifier, and optionally less than about 1 wt. % stabilizer or other additive.

While equipment has been designed to supply and apply hot-melt adhesives, and various manufacturing processes have been designed to accommodate and utilize hot-melt adhesives in the manufacture of articles, these adhesives are accompanied by challenges (some of which are discussed herein). For example, in the absence of a viscosity modifier, such adhesives typically melt and, therefore, must be applied at a temperature of about 150° C. to about 190° C. Materials sensitive to such temperatures, of course, cannot be adhered with such adhesives. Disposable absorbent articles typically contain these temperature-sensitive materials. And, in the manufacture of such articles, the manufacturing processes must accommodate adhesives suitable for application to such materials, in addition to the conventional hot-melt adhesive. There would be benefits in reducing the number and types of adhesives used to construct such articles. Furthermore, there would be benefits in providing these processes with an adhesive that need not be heated to the high temperatures required of a conventional hot-melt adhesive.

The art has attempted to address this challenge by incorporating into the hot-melt adhesive free ingredients that will modify the melt temperature of the adhesive composition. These ingredients effectively increase the fluidity or plasticity and modify the viscosity of the composition to which they are added and, hence, are commonly referred to as plasticizers. A common plasticizer is mineral oil. But while the presence of free plasticizers in hot-melt adhesive compositions improves the fluidity and reduces the melt temperature of these compositions, the free plasticizers also are, of course, additional ingredients of the composition. Accordingly, the cost of preparing the composition and blending the ingredients can be expected to increase concomitantly with additional ingredients.

Aside from the costs associated with additional ingredients, free plasticizers can undesirably migrate and/or leach into components of the article intended to be joined together by the adhesive. That migration and/or leaching at the manufacturing stage results in articles unsuitable for sale to a consumer because, for example, the plasticizer may compromise the wicking and wetting abilities of the article. Further, migration (e.g., blooming) and/or leaching following the manufacturing stage, for example during storage, can result in articles that are not comfortable to the end user (e.g., infants in the case of infant diapers). Over time, and even following a single bad experience, consumers may not purchase or use such a product again. The art, however, has not addressed how to rid hot-melt adhesive compositions of the free plasticizer and, therefore, the potential problems associated with the presence of free plasticizer persist.

One response to the problems associated with free plasticizers in hot-melt adhesive compositions has been to employ an adhesive composition that fundamentally differs from hot-melt adhesives. Conventional hot-melt adhesives, in fluidic form, harden and form adhesive bonds upon cooling below either a melt temperature or the glass transition temperature. In contrast, the art has recently developed adhesives containing reactive alkyd resins, solvents, and other ingredients.

Alkyd resins are thermosetting polymers, chemically similar to polyester resins, typically made by condensation and polymerization of a polyhydric alcohol with an acid or acid anhydride. Reactive alkyd resins, in fluid form, become gel-like and form adhesive bonds by covalent bonds that form as a result of the condensation and polymerization reactions. Desirably, relative to hot-melt adhesive compositions, compositions containing reactive alkyd resin do not need plasticizers. The gel-formation (and, therefore, adhesive bond formation), however, is irreversible in that the material may not be melted to form a flowable material and then re-cooled to form an adhesive bond. More importantly, adhesive compositions employing reactive alkyds in the manufacture of disposable absorbent articles must be prepared and applied at temperatures far higher than that at which conventional hot-melt adhesives can be applied. For example, the temperature at which reactive alkyd resins form and are used in the context of adhesive compositions is 150° C. to about 220° C. Temperature-sensitive materials (e.g., polyolefins) that are common components in the manufacture of disposable absorbent articles cannot withstand these temperatures. Consequently, these adhesives are not necessarily an ideal substitute for conventional hot-melt adhesives. Alternatively, the use of lower processing temperatures will require longer processing periods than is customary when using conventional hot-melt adhesives. Any increase in processing periods, in turn, requires modifications to the manufacturing process and diminished production yields.

Generally, an adhesive should have a surface energy no greater than and, preferably, matching, that of the substrates sought to be adhered. That relationship ensures strong adhesion (mechanically) between the adhesive and the substrate. The surface energy of conventional construction adhesives, however, is not readily adjustable. Consequently, with a set surface energy, conventional construction adhesives can suitably adhere a somewhat limited class of substrates together. There would be benefits in employing a construction adhesive whose surface energy is capable of modification to better match the surface energies of substrates.

Conventional hot-melt adhesives include ingredients that are directly dependent upon components of crude-oil refining. As expenses associated with the latter increase, costs in employing the former increase. Consequently, there would be benefits in employing construction adhesives that contain fewer such ingredients, reduced amounts of such ingredients, and possibly no such ingredients. At the same time, there would be, of course, environmental and sustainability benefits in employing construction adhesives that contain fewer such ingredients, reduced amounts of such ingredients, and possibly no such ingredients.

The foregoing discussion simply highlights some of the challenges that accompany the use of conventional construction adhesives.

### SUMMARY OF THE INVENTION

Disclosed herein is a new use for water-stable, oil-modified, nonreactive alkyd resins. It has now been found that such resins can be used as the predominant component of a construction adhesive. Accordingly, one aspect of the invention disclosed herein, includes such adhesives. And, as explained in more detail below, these adhesives address one or more of the above-described challenges associated with conventional construction adhesives. On the basis of this finding, disclosed herein are additional aspects of the invention directed to articles that employ these alkyd resin-containing construction adhesives, and methods of making the articles.

A construction adhesive composition includes a water-stable, oil-modified, nonreactive alkyd resin. More specifically, the composition includes at least about 65 wt. %, based on the total weight of the composition, of a water-stable, oil-modified, nonreactive alkyd resin. Furthermore, the composition includes about 0.01 wt. % to about 35 wt. %, based on the total weight of the composition, of one of (i) a polymer selected from the group consisting of polyhydroxyalkanoates, polyvinyl alcohol, polyethylene, polypropylene, polyethylene terephthalate, maleated polyethylene, maleated polypropylene, polylactic acid, modified polypropylene, nylon, caprolactone, and combinations thereof; and, (ii) a processing aid selected from the group consisting of talc, clay, pulp, flour, walnut shells, cellulose, cotton, jute, raffia, rice chaff, animal bristles, chitin, granular starch, diatomaceous earth, carbon fibers, kenaf, magnesium stearate, fatty acid amides, metal salts of fatty acids, wax acid esters and their soaps, montan wax acids, esters and their soaps, polyolefin waxes, non polar polyolefin waxes, natural and synthetic paraffin waxes, fluoro polymers, silicon, diatomaceous earth, and combinations thereof.

An article, for example a disposable absorbent article, according to an aspect of the invention includes disposable absorbent article components, and an adhesive composition applied between and joining at least two of the disposable adsorbent article components together. As described in further detail below, the adhesive composition includes a water-stable, oil-modified, nonreactive alkyd resin.

Another aspect of the invention is a method of forming an adhesive bond between components of an article, such as a disposable absorbent article. The method generally includes applying to a first article component an adhesive composition that includes a water-stable, oil-modified, nonreactive alkyd resin at a temperature of about 80° C. to about 200° C., and joining the first article component with a second article component to form an adhesive bond as the adhesive cools to room temperature.

Although the articles are not limited to consumer products falling within specific categories, a representative, non-limiting list of such categories includes baby care, feminine protection, incontinence care, paper products, and household care articles. A variety of articles may fall within these categories. Exemplary baby care articles include diapers, wipes, baby bibs, baby change and bed mats. Exemplary feminine protection articles include pads, tampons, interlabial products and pantyliners. Exemplary incontinence care articles include diapers, pads, and liners. Exemplary paper products include toilet tissue, paper towels, and facial tissues. Exemplary household care articles include sweeper products, and floor cleaning articles. As described in further detail below, the articles can include a variety of personal hygiene products such as, for example, absorbent articles including diapers, sanitary napkins, tissues, towels, and wipes, and also non-hygiene products such as, for example, packaging articles and containers.

Additional features of the invention may become apparent to those skilled in the art from a review of the following detailed description, taken in conjunction with the drawings, the examples, and the appended claims.

### BRIEF DESCRIPTION OF THE DRAWINGS

For a more complete understanding of the disclosure, reference should be made to the following detailed description and accompanying drawing wherein:

FIG. 1 is a partially cut-away perspective view of an embodiment of a disposable absorbent article, specifically a diaper, incorporating an alkyd resin-containing construction adhesive;

FIG. 2 is a correlational analysis plotting wavelength versus absorption intensity (arbitrary units) of an ATR/IR spectra obtained by stepwise heating and cooling, and a water-stable, oil-modified, nonreactive alkyd resin;

FIG. 3 is a graph depicting the dynamic viscosity (η) over a temperature range of various grades of an alkyd resin; and,

FIG. 4 is a graph depicting the elastic or storage modulus (G′) and the viscous or loss modulus (G″) over a temperature range for a conventional hot-melt adhesive and an alkyd resin.

While the specification concludes with claims particularly pointing out and distinctly claiming the subject matter that is regarded as the present invention, it is believed that the invention will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

### DETAILED DESCRIPTION OF THE INVENTION

It has now been found that water-stable, oil-modified, nonreactive alkyd resins can be used as the predominant component of a construction adhesive. These resins have been employed in the manufacture of paints. Heretofore, however, these resins were not employed as a predominant ingredient in construction adhesives. Accordingly, compositions containing (and, preferably, predominantly containing) water-stable, oil-modified, nonreactive alkyd resins are capable of addressing one or more of the above-described challenges associated with conventional construction adhesives.

Disclosed and explained in further detail herein are these alkyd resin-containing construction adhesives, articles that include these adhesives, and methods of making the articles. As described in further detail below, the articles can include a variety of personal hygiene products such as, for example, absorbent articles including diapers, sanitary napkins, tissues, towels, and wipes, and also non-hygiene products such as, for example, packaging articles and containers.

Generally, an absorbent article (e.g., a disposable absorbent article) according to an aspect of the invention includes absorbent article components, and an adhesive composition applied between and joining at least two of the adsorbent article components together. As described in further detail below, the adhesive composition includes a water-stable, oil-modified, nonreactive alkyd resin. Another aspect of the invention is a method of forming an adhesive bond between components of an article, such as a disposable absorbent article. The method generally includes applying to a first article component an adhesive composition that includes a water-stable, oil-modified, nonreactive alkyd resin at a temperature of about 80° C. to about 150° C., and joining the first article component with a second article component to form an adhesive bond as the adhesive cools to room temperature.

The alkyd resin-containing adhesives described herein are particularly beneficial and address long-felt needs in the art concerned with manufacturing disposable absorbent articles, such as diapers and sanitary napkins. Specifically, and as mentioned above, this art has long employed hot-melt adhesives that contain free plasticizers. Those free plasticizers often undesirably migrate and/or leach into components of the article intended to be joined together by the adhesive. That migration and/or leaching at the manufacturing stage results in articles unsuitable for sale to a consumer because, for example, the plasticizer may compromise the wicking and wetting abilities of the article. Further, migration (e.g., blooming) and/or leaching following the manufacturing stage, for example during storage, can result in articles that are not comfortable to the end user (e.g., infants in the case of infant diapers). Over time, and even following a single bad experience, consumers may not purchase or use such a product again.

Furthermore, the alkyd resin-containing adhesives described herein provide a number of other benefits over hot-melt adhesives typically used as construction adhesives. Specifically, the alkyd resin-containing adhesives described herein are of sufficiently low viscosity at low processing temperatures and, therefore, can be applied to bond substrates at a lower temperature than can conventional hot-melt adhesives. This results in reduced operating costs in that the particular adhesive now need not be heated to the high temperatures required in the prior art. At the same time, the processing equipment presently employed to apply conventional hot-melt adhesives in article manufacturing processes need not be exchanged for new equipment. This results in no further expenses in capital costs.

Still further, the alkyd resin-containing adhesives described herein have a melt viscosity substantially lower than that of conventional hot-melt adhesives. The lower melt viscosity translates into increased fluidity at a given temperature and, importantly, at the lower processing temperatures at which the adhesive can be applied relative to conventional hot-melt adhesives. This makes application of the adhesive easier in that many more applicators can be considered, whereas the group of potential applicators was previously limited by the properties and characteristics of conventional hot-melt adhesives. Furthermore, the lower melt viscosity also broadens the scope of substrates that can be bonded together with the adhesive. Specifically, the substrate components bound by the adhesive can be selected more freely because the temperature processing range of the adhesive is more broad and includes a low processing temperature (relative to conventional hot-melt adhesives) range necessary for binding low melting-point materials, such as polyolefins and other temperature-sensitive substrates. As explained in more detail herein, that scope is further narrowed because conventional hot-melt adhesives typically have a surface energy incompatible with a broader scope of substrates.

The alkyd resin-containing adhesives described herein are advantageous because the surface energy thereof can be readily adjusted. Generally, an adhesive should have a surface energy no greater than, and preferably matching, that of the substrates sought to be adhered. That relationship ensures strong adhesion (mechanically) between the adhesive and the substrate. The surface energy of conventional construction adhesives, however, is not readily adjustable. Consequently, the scope of substrates that could be adjusted by any given construction adhesive was necessarily limited on the basis of the surface energy of the adhesive. The ability to adjust the surface energy of the alkyd resin-containing adhesives disclosed herein now makes possible the ability to adhere a broader scope of substrates.

Furthermore, the alkyd resin-containing adhesives described herein can be made much more cost-efficiently than conventional hot-melt adhesives. The latter typically require the presence of a polymer to impart cohesive forces to the adhesive. That polymer is often manufactured by an energy-intensive process, the operating costs of which are tied to external factors, namely the costs of crude oil and crude oil refining. The alkyd resin-containing adhesives described herein, however, are not tied to such external factors. Instead, and beneficially, the alkyd resin-containing adhesives described herein can be manufactured from low-cost, renewable sources, such as fatty acids and vegetable oils.

These and other benefits will become more apparent from the following description.

“Absorbent article” is used herein to refer generally to a device that absorbs and contains liquid. One class of such articles includes a device that is placed against or in proximity to a device wearer's body to absorb and contain various exudates discharged from the body. That class of absorbent articles includes items such as diapers, pull-on diapers, pant-type garments, training pants, incontinence briefs, incontinence undergarments, absorbent inserts, diaper holders and liners, feminine hygiene garments (e.g., sanitary napkins), and the like. Another class of absorbent articles includes a device that is applied to absorb, contain, or clean various liquids. That class of absorbent articles includes paper-based items such as napkins, tissues (e.g., facial and toilet tissues), towels, and wipes.

“Construction adhesive” is used herein to refer generally to an adhesive used to join one or more components of an absorbent article to themselves or together during the manufacturing process. The adhesive bond formed by a construction adhesive is typically intended to be a permanent bond, but need not necessarily be so.

“Disposable” is used herein to describe absorbent articles that generally are not intended to be laundered or otherwise restored or reused as an absorbent article (i.e., articles intended to be discarded after a single use and, possibly, to be recycled, composted or otherwise discarded in an environmentally compatible manner).

“Pant-type” is used herein to refer to an article configured such that it has a waist opening and a pair of leg openings. A pant may be placed in position on the wearer by inserting the wearer's legs into the leg openings and sliding the pant into position about a wearer's lower torso. This configuration may be permanent as in the case of conventional underwear, or may be temporary as in the case of a training pant with openable seams for removal. Additionally, absorbent articles can be constructed with refastenable features allowing the article to have both a pant-like configuration and one or more configurations which are open or not pant like.

“Longitudinal” is used herein to refer to a direction running perpendicular from a waist edge to an opposing waist edge of the article and generally parallel to the maximum linear dimension of the article. Directions within 45 degrees of the longitudinal direction are considered to be “longitudinal.”

“Lateral” is used herein to refer to a direction running from a side edge to an opposing side edge of the article and generally at a right angle to the longitudinal direction. Directions within 45 degrees of the lateral direction are considered to be “lateral.”

As used herein, the terms “elastic,” “elastomer,” “elastomeric,” “elastically,” and “stretchable” generally refer to materials which are extensible by a pulling force, and which also return to substantially their original dimensions when the external pulling force is removed. More specifically, these terms refer to a material that is able to extend to a strain of at least 50% without breaking or rupturing, and is able to recover substantially to its original dimensions after the deforming force has been removed. It will be appreciated that these terms include the term “extensible” as each term is used herein.

“Garment-facing” is used herein to refer to describe a surface that is in contact with or may be in close proximity to any garment being worn.

“Body-facing” is used herein to refer to describe a surface that is in contact with the body of a wearer or in close proximity (i.e., closer to the body than a garment-facing surface) to the body of the wearer when the article is worn.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

The term “fixed,” as used when referring to elements that are “fixed” to one another, means the elements are ordinarily joined or attached together by the manufacturer of the article in a manner such that the wearer or user of the article will not be able to un-join or detach the elements during the article's ordinary use, and the elements will not become un-joined or detached through the article's ordinary wear and tear. Elements that are “fixed” to one another are not intended to be separated during normal use of the article.

As used herein, the terms “refastenable,” “releasably fastenable,” and “engageable” refer to attachment of two or more elements or portions of elements together in a manner in which they can be separated and re-attached without substantial degradation of fastener performance or damage to surrounding components of the article which would impair the article's continued use. It will be appreciated that a refastenable, releasably fastenable, or engageable component need not have an infinite life span, but it is sufficient that the components attached in a refastenable, releasably fastenable, or engageable manner can be separated and re-attached successively several times over the typical use life span of the article. It will also be appreciated that the aggressiveness of actual fastening may be reduced significantly from fastening to refastening in absolute terms, but that such reduction is not “substantial degradation” of fastener performance if the resulting refastened strength is sufficient for purposes of ordinarily using the article and fastener.

“Mechanical fastener” is used herein to refer to a fastening system or mechanism relying on physical restraint, magnetic fields, or engagement of portions of the fastener for operation. Examples of mechanical fasteners are hook and loops, hooks and hooks, buttons, snaps, tab and slot, zippers, magnet(s), and tongue and groove fasteners.

“Spiral” is used herein to refer to a shape or pattern that generally resembles a helix as viewed in a two dimensional plane from a direction substantially orthogonal to the helix's axis of rotation. The construction adhesives disclosed herein can be applied via a spiral applicator to adhere components of an absorbent article to one another.

“Adhesive” and an “adhesive composition” are used herein to generally refer to a material that joins two other materials, called adherends, together. These materials are applied as a liquid, preferably of a low viscosity. The liquid form is obtained by heating the material to a point that flow occurs. In the liquid form, the material is applied to the adherend(s) and wets and flows into the crevices (if any) of the adherend(s). The material then undergoes a phase change to a solid by cooling in order for the joint to acquire the necessary strength to resist shearing forces.

The alkyd resin-containing adhesive generally includes a water-stable, oil-modified, nonreactive alkyd resin. In a preferred embodiment, the alkyd resin is present in the adhesive composition in an amount of at least about 65 wt. % based on the total weight of the adhesive composition, alternatively at least about 80 wt. % or at least about 90 wt. %, based on the total weight of the adhesive composition. In further alternative embodiments, the alkyd resin may be present in the aforementioned amounts, but less than 100 wt. %, based on the total weight of the adhesive composition. In another alternative embodiment, the adhesive can be made up entirely of this alkyd resin. Accordingly, and in certain of the alternative embodiments, the adhesive composition can be free of free plasticizers and free of free tackifiers.

“Water-stable” is used herein to refer to the alkyd resin's ability to withstand degradation by water or other aqueous materials under the conditions the resin can be expected to encounter as an adhesive component in the manufacture and use of the articles described herein. Thus, in the context of a diaper, for example, an alkyd resin forming a component of an adhesive used to construct the diaper is water stable if it is capable of maintaining its chemical structure and viscosity after having been exposed to body exudates, including blood, urine, and other aqueous materials for a period of about 3 to 36 hours, preferably a period of about 6 to about 18 hours, and more preferably a period of about 8 to about 12 hours. Under these exposure periods, a water-unstable product can be expected to dissolve or otherwise wash away from its adherends. In contrast, a water-stable material should not do so. Water stability in the context of an adhesive containing the alkyd resin can be more quantitatively determined by a Bond Strength test described in more detail below. Generally, the adhesive containing the alkyd resin is water stable, according to the Bond Strength test, if the bond strength of an article employing the adhesive experiences a bond strength loss of less than 20% after the article has been exposed to ambient-temperature water for at least 16 hours.

“Nonreactive” is used herein to refer to the chemical stability of the alkyd resin. Specifically, a nonreactive alkyd resin neither chemically reacts nor requires chemical reaction during application or dispensation of the resin in a manner that would affect the resin's ability to function as an adhesive or a predominant component of an adhesive. For example, nonreactive alkyd resins for use in accordance with the invention undergo no chemical reaction (other than perhaps some incidental reactions, such as minor cross linking or oxidation) when exposed to the processing temperatures herein contemplated of an adhesive. Furthermore, nonreactive is used herein to denote that the alkyd resin experiences no appreciable changes in rheological properties, adhesive strength, cohesive strength, specific heat, or surface energy when exposed to the processing temperatures herein contemplated of an adhesive. Nonreactive is further used herein to distinguish the reactive alkyds described above.

As generally explained above, an alkyd resin is the product of a reaction between a polyhydric alcohol and an acid or acid anhydride. In accordance with the invention disclosed herein, the alkyd resins are modified with fatty acids, saturated or unsaturated (preferably from plant and vegetable oils). Consequently, “oil-modified” is used herein to refer to an alkyd resin containing fatty acid (oil) modification of the resin. And, in preferred embodiments, the alkyd resin is an ester condensate of polyhydric alcohols, organic polyacids, and vegetable oils. The components of the water-stable, oil-modified, nonreactive alkyd resin can be combined in any manner known by those having ordinary skill in the art. For example, the components can be combined by mixing the same at a temperature of about 200° C. to about 250° C. for a time period sufficient to form the alkyd resin.

As used herein, “polyhydric alcohol” refers to an alcohol having two or more alcohol (i.e., hydroxyl) functional groups. Any suitable polyhydric alcohol or combination of polyhydric alcohols may be used in accordance with the invention. Non-limiting examples of suitable polyhydric alcohols include glycerol, glycol, sugar, sugar alcohol, and combinations thereof. Non-limiting examples of glycols include ethylene glycol, propylene glycol, dipropylene glycol, butylene glycol, hexane triol, dimethylol pentane, dimethylol ethane, and the like, polymers thereof, and combinations thereof. Non-limiting examples of sugars include glucose, sucrose, fructose, raffinose, maltodextrose, galactose, xylose, maltose, lactose, mannose, erythrose, pentaerythritol, dipentaerythritol and mixtures thereof. Non-limiting examples of sugar alcohols include erythritol, xylitol, malitol, mannitol, sorbitol, and mixtures thereof. In certain specific embodiments, the polyhydric alcohol includes glycerol, mannitol, sorbitol, and combinations thereof.

Generally, the polyhydric alcohol is substantially compatible with any polymeric components with which it may be intermixed. As used herein, “substantially compatible” means that the polyhydric alcohol is capable of forming a visually homogeneous mixture with polymer present in the composition in which it is intermixed when the polyhydric alcohol is heated to a temperature above the softening and/or the melting temperature of the composition.

In the manufacture of suitable alkyd resins, the polyhydric alcohol can be present in a reaction mixture in an amount of from about 2 wt. % to about 70 wt. % from about 5 wt. % to about 50 wt. %, from about 10 wt. % to 30 wt. %, or from about 15 wt. % to about 25%.

Suitable acids have at least one functional group selected from the group consisting of carboxylic acid, carboxylic acid anhydride, and combinations thereof. Non-limiting examples of classes of such acids include monoacids, diacids, polyacids (acids having at least three acid group), polymers comprising at least one acid moiety, co-polymers comprising at least one acid moiety, anhydrides thereof, and mixtures thereof. More specifically, non-limiting examples of such acids include adipic acid, sebatic acid, lauric acid, stearic acid, myristic acid, palmitic acid, oleic acid, linoleic acid, sebacic acid, citric acid, oxalic acid, malonic acid, succinic acid, glutaric acid, maleic acid, fumaric acid, phthalic acid, isophthalic acid, terephthalic acid, acrylic acid, methacrylic acid, itaconic acid, glycidyl methacrylate, and combinations thereof.

Anhydrides of such acids also may be employed within the context of the present invention. Non-limiting examples of acid anhydrides include maleic anhydride, phthalic anhydride, succinic anhydride, and combinations thereof.

Polymers and co-polymers that include at least one acid moiety, and/or their anhydrides also may be used. Non-limiting examples of suitable polymers and copolymers include those having monomer units of acrylic acid, methacrylic acid, itaconic acid, glycidyl methacrylate, anhydrides thereof, and combinations thereof. The polymer can contain other monomer units in conjunction with these acid monomer units. For example, ethylene-acid monomer copolymers such as ethylene-acrylic acid copolymer can be used. In a specific embodiment, the copolymers include at least 50 mole % of acid monomer units. The molecular weight of such polymers and copolymers can vary from as low as about 2,000 to over about 1,000,000. An example of a suitable polyacrylic acid is one having a molecular weight of about 450,000 (available as Product No. 181285 from Aldrich Chemical Company, Inc., Milwaukee, Wis.). An example of a suitable ethylene-acrylic acid copolymer is Primacore 59801 (available from The Dow Chemical Company, Midland, Mich.), which has an acrylic acid content of at least 50 mole %.

In specific embodiments, the acid includes at least one diacid, polyacid, acid polymer or copolymer, or a mixture thereof. In other embodiments, the acid includes a diacid, alone or in combination with another acid, for example a monoacid. In further embodiments, the acid includes adipic acid, stearic acid, lauric acid, citric acid, polyacrylic acid and/or ethylene-acrylic acid copolymer. Preferred polyacids include aromatic acids such as phthalic acid, and preferred polybasic acid anhydrides include phthalic anhydride or trimellitic anhydride.

In the manufacture of suitable alkyd resins, the acid is employed in the reaction mixture in an amount of from about 0.1 wt. % to about 30 wt. %, from about 1 wt. % to about 20 wt. %, or from about 2 wt. % to about 12 wt. %. In some embodiments, the molar ratio of alcohol functional groups to acidic functional groups in the composition is at least about 1:1, or at least about 4:1. In some embodiments, the molar ratio of alcohol functional groups to acidic groups in the composition is from about 1:1 to about 200:1,or from about 1:1 to about 50:1.

Suitable oils for modifying the alkyd resin include triglycerides such as, for example, tristearin, triolein, tripalmitin, 1,2-dipalmitoolein, 1,3-dipalmitoolein, 1-palmito-3-stearo-2-olein, 1-palmito-2-stearo-3-olein, 2-palmito-1-stearo-3-olein, trilinolein, 1,2-dipalmitolinolein, 1-palmito-dilinolein, 1-stearo-dilinolein, 1,2-diacetopalmitin, 1,2-distearo-olein, 1,34stearo-olein, trimyristin, trilaurin and combinations thereof.

Suitable triglycerides should be added to the reaction mixture from which the alkyd resin is formed. Additionally, or alternatively, oils and/or processed oils containing suitable triglycerides can be added to that mixture. Non-limiting examples of oils include beef tallow, castor oil, coconut oil, coconut seed oil, corn germ oil, cottonseed oil, fish oil, linseed oil, olive oil, oiticica oil, palm kernel oil, palm oil, palm seed oil, peanut oil, rapeseed oil, safflower oil, soybean oil, sperm oil, sunflowerseed oil, tall oil, tung oil, whale oil, and mixtures thereof. Unsaturated and saturated fatty acids may be suitable. Non-limiting examples of such fatty acids include capric acid, caproic acid, caprylic acid, lauric acid, lauroleic acid, linoleic acid, linolenic acid, myristic acid, myristoleic acid, oleic acid, palmitic acid, palmitoleic acid, stearic acid, and mixtures thereof. Combinations of the aforementioned oils and fatty acids also can be employed to modify the alkyd resin.

In the manufacture of suitable alkyd resins, triglycerides are present in the reaction mixture in an amount of from about 0.1 wt. % to about 70 wt. %. In certain embodiments, the amount of triglycerides present in the reaction mixture is from about 56 wt. % to about 70 wt. %, alternatively from about 46 wt. % to about 55 wt. %, and alternatively in an amount of less than 45 wt. %, such as, from about 1 wt. % to about 20 wt. %, or from about 2 wt. % to about 12 wt. %. In some embodiments, the molar ratio of alcohol functional groups to ester functional groups in the composition is at least about 1:1, or at least about 4:1. In some enibodiments, the molar ratio of alcohol tunctional groups to ester functional groups in the composition is from about 1:1 to about 200:1, or from about 1:1 to about 50:1.

In some embodiments, combinations of acid and triglyceride are present in the reaction mixture for the manufacture of the alkyd resin. In some embodiments, the total amount of acid and triglyceride present in the reaction mixture is from about 0.1 wt. % to about 70 wt. %. In certain embodiments, the total amount of acid and triglyceride present in the reaction mixture is from about 56 wt. % to about 70 wt. %, alternatively from about 46 wt. % to about 55 wt. %, and alternatively in an amount of less than 45 wt. %, such as, from about 1 wt. % to about 25 wt. %, or from about 2 wt. % to about 20 wt. %. Additionally, or alternatively, the molar ratio of the alcohol functional groups to the total of ester and acid functional groups is at least about 1:1, or at least about 4:1. In some embodiments, the molar is from about 1:1 to about 200:1, or from about 1:1 to about 50:1.

Without wishing to be bound by any particular theory, it is believed that oil modification to the alkyd resin imparts the resin with a number of properties that render it advantageous for use as an adhesive and as the predominant component of a construction adhesive. For example, it is believed the oil modification imparts internally (by way of covalent bonding) to the resin the function of a plasticizer. Furthermore, it is believed, without wishing to be bound by any particular theory, that alkyd resins with a higher content of oil (e.g., a long-oil alkyd resin) are likely to have stronger characteristics of a plasticizer. The oil modification to the alkyd resin, imparting the resin with an internal plasticizer (by covalent bonding), obviates the need to include a free plasticizer in an adhesive composition. In turn, that avoids the potential problems associated with using a free plasticizer.

Furthermore, without wishing to be bound by any particular theory, it is believed that oil modification to the alkyd resin imparts to the resin improved adhesive strength properties and improved hydrophobicity that, in turn, improves the cohesive strength properties of the resin. These improved properties are believed to contribute to the resin's ability to withstand degradation to water or other aqueous liquids.

The adhesive compositions may include one or more additional components, as may be desired for the processing and/or end use of the composition, though these components are not required. Additional components may be present in any suitable amount. In some embodiments, additional components may be present in an amount of from about 0.01 wt. % to about 35 wt. % or from about 0.1 wt. % to about 20 wt. %, or from about 0.1 wt. % to about 10 wt. %, based on the total weight of the adhesive composition. Non-limiting examples of additional components include, but are not limited to, additional polymers, processing aids, and the like.

Non-limiting examples of additional polymers include polyhydroxyalkanoates, polyvinyl alcohol, polyethylene, polypropylene, polyethylene terephthalate, maleated polyethylene, maleated polypropylene, polylactic acid, modified polypropylene, nylon, caprolactone, and combinations thereof.

In embodiments in which properties including, but not limited to, biodegradability and/or flushability are desired, suitable biodegradable polymers and combinations thereof may be used. In some embodiments, polyesters containing aliphatic components are suitable biodegradable thermoplastic polymers. In some embodiments, among the polyesters, ester polycondensates containing aliphatic constituents and poly(hydroxycarboxylic) acid are preferred. Non-limiting examples of the ester polycondensates include diacids/diol aliphatic polyesters such as polybutylene succinate, and polybutylene succinate co-adipate; aliphatic/aromatic polyesters such as terpolymers made of butylenes diol, adipic acid, and terephthalic acid. Non-limiting examples of the poly(hydroxycarboxylic) acids include lactic acid based homopolymers and copolymers; polyhydroxybutyrate; and other polyhydroxyalkanoate homopolymers and copolymers. In some embodiments, a homopolymer or copolymer of polylactic acid is preferred. Modified polylactic acid and different stereo configurations thereof may also be used. Suitable polylactic acids typically have a molecular weight range of from about 4,000 g/mol to about 400,000 g/mol. Non-limiting examples of suitable commercially-available polylactic acids include NATUREWORKS™ (available from NatureWorks LLC, Minnetonka, Minn.) and LACEA™ (available from Mitsui Chemicals, Inc., Tokyo, Japan). Non-limiting examples of suitable commercially-available diacid/diol aliphatic polyester include polybutylene succinate/adipate copolymers, such as BIONOLLE™ 1000 and BIONOLLE™ 3000 (available from the Showa Highpolmer Company, Ltd., Tokyo, Japan). An example of a suitable commercially available aliphatic/aromatic copolyester is poly(tetramethylene adipate-co-terephthalate), such as EASTAR BIO™ Copolyester (available from Eastman Chemical, Kingsport, Tenn.) and ECOFLEX™ (available from BASF, Ludwigshafen, Germany). In some embodiments, the biodegradable polymer or combination of polymers may include polyvinyl alcohol. The biodegradable polymers and combinations thereof may be present in an amount of less than about 10 wt. %, based on the total weight of the adhesive composition.

Processing aids (including fillers) may generally be present in the compositions in amounts of from about 0.1 wt. % to about 3 wt. %, or from about 0.2 wt. % to about 2 wt. %. Non-limiting examples of processing aids include lubricants, anti-tack, polymers, surfactants, oils, slip agents, and combinations thereof. Non-limiting examples of specific processing aids include talc, clay, pulp, flour, walnut shells, cellulose, cotton, jute, raffia, rice chaff, animal bristles, chitin, granular starch, diatomaceous earth, carbon fibers, kenaf, magnesium stearate, fatty acid amides, metal salts of fatty acids, wax acid esters and their soaps, montan wax acids, esters and their soaps, polyolefin waxes, non polar polyolefin waxes, natural and synthetic paraffin waxes, fluoro polymers, silicon, and combinations thereof. Commercial examples of such compounds include, but are not limited to: Crodamide™ (Croda, North Humberside, United Kingdom), Atmer™ (Uniqema, Everberg, Belgium,) and Epostan™ (Nippon Shokobai, Tokyo, Japan).

The aforementioned additional components of the water-stable, oil-modified alkyd resin can be combined in any manner known by those having ordinary skill in the art. For example, and consistent with the description set forth above, these additional components can be combined with the reaction mixture that forms the alkyd resin by mixing the same at a temperature of about 200° C. to about 250° C. for a time period sufficient to form the alkyd resin.

The water-stable, oil-modified, nonreactive alkyd resins used in the adhesive impart the adhesive with thermal stability sufficient for the applications in which the adhesive is herein contemplated. The temperature range in which the adhesive can be expected to be applied ranges from about 80° C. to about 200° C. For example, in specific applications, preferred adhesive application temperatures for polyethylene is about 90° C. to about 110° C., for polypropylene it is about 120° C. to about 150° C., for cellulosics it is about 100° C. to about 130° C., and for polyethylene terephthalate it is about 140° C. to about 160° C. Within that range, the alkyd resins contemplated for use as a construction adhesive herein are expected to maintain their chemical composition (e.g., no pertinent reaction or degradation) and physical properties, such as rheological properties (e.g., substantially constant viscosity).

Furthermore, the water-stable, oil-modified, nonreactive alkyd resins used in the adhesives and, therefore, the adhesives themselves, can be designed to have a surface energy that matches the particular substrates intended to be adhered. For example, it is possible to modify the surface energy of these resins and thereby the surface energy of the adhesives, to provide a surface energy of at least about 40 dynes per centimeter (dynes/cm), which is desirable when adhering together components (e.g., two plies of a tissue) of absorbent articles such as paper towels, napkins, wipes, and handkerchiefs. Components of these articles typically have a surface energy of at least about 40 dynes/cm. Without wishing to be bound by any particular theory, it is believed that the presence of free acid groups and/or free alcohol groups on the polymeric backbone or chain of the alkyd resin is determinative of the resin's surface energy. The greater the number of free acid-groups and/or free alcohol-groups, the greater the surface energy. And alkyd resins can be controllably designed by those having skill in the art to have a specified number of free acid-groups and/or free alcohol-groups. For example, to reduce the surface energy, these groups can be capped during the resin's manufacture with capping agents known by those having ordinary skill in the art of alkyd resin manufacture. Capping agents generally include monofunctional acids, such as benzoic acid and acetic acid, or monofunctional alcohols. The capping of these groups is not believed to detrimentally affect other properties of the resin that make the resin suitable for use as a construction adhesive.

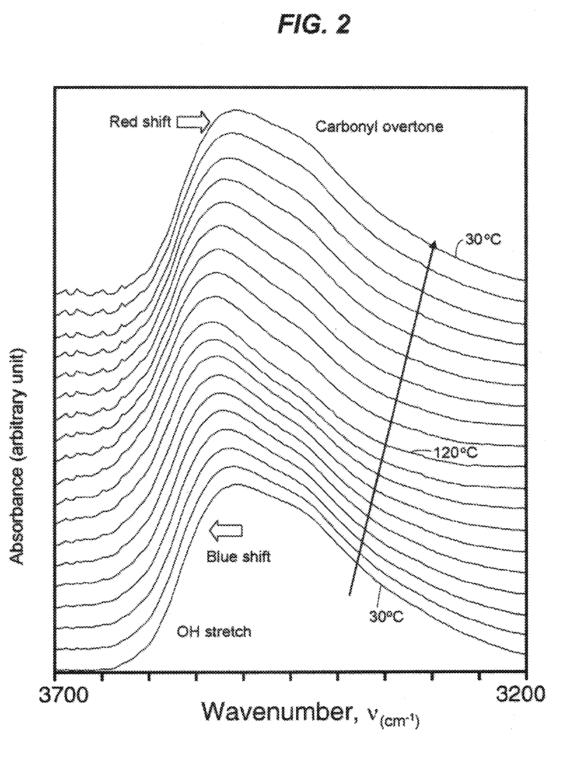

The water-stable, oil-modified, nonreactive alkyd resins that predominantly comprise the construction adhesive disclosed herein behave different than how a conventional hot-melt adhesive behaves when exposed to a melt temperature. Shown in FIG. 2 is a correlational analysis plotting wavelength versus absorption intensity (arbitrary units) of an ATR/IR spectra obtained by stepwise heating and cooling (from 30° C. to 120° C. to 30° C.) a water-stable, oil-modified, nonreactive alkyd resin. The resin is solvent free, and is otherwise believed to be similar to one commercially available from Reichhold Chemicals Inc. (Durham, N.C.). under the trade name BECKOSOL® 12-035, No. EM-193203. The plotted data show that hydrogen bonding is an important factor in determining the fluidity of the resin over the process temperature range. Generally, hydroxyl (OH) stretching vibration frequency shifts to a lower wavenumber region (Red shift) if the hydroxyl group is hydrogen bonded. The breaking of the hydrogen bond by beating results in a higher wavenumber (Blue) shift. See generally, George C. Pimentel et al., “The Hydrogen Bond,” 68-79 (Reinhold Publishing Company, New York, 1960). In FIG. 2, a noticeable and significant Blue shift of the hydroxyl (OH) stretching peak upon heating is indicative of hydrogen bonding interactions within the alkyd matrix.

The plot further reveals the presence of the emerging free hydroxyl group among the dominant hydrogen-bonded hydroxyl vibrations, as well as a significant contribution from the overtone of carbonyl stretching absorption. These data suggest that the dramatic change in the rheological properties of the oil-modified alkyd resin is caused not by the conventional mechanism of melting crystals or the polymeric solidification transition. Without wishing to be bound by any particular theory, it is believed that the rheological property change is attributed to the resin undergoing a temperature-induced gel-to-sol transition. This is supported by the data obtained when the material is cooled (from 120° C. to 30° C.). There, the population of the highly-associated hydroxyl groups observed at lower wavenumber region increases first, followed by the eventual disappearance of the free hydroxyl groups. The spectra suggest the network of hydrogen-bonded solid upon heating is converted to liquid containing some broken-up, free hydroxyl groups. The thermally-induced disruption and reformation of the hydrogen bonding network appears completely reversible. This type of transition also provides much more gradual changes in the mechanical properties than, for example, a sudden melting of crystals. The more gradual change is advantageous because it provides a broader process window for adhesives containing these resins.

Suitable water-stable, oil-modified, nonreactive alkyd resins are commercially available from Reichhold Chemicals Inc. (Durham, N.C.), under the trade name BECKOSOL®, and also from Hexion Specialty Chemicals, Inc. (Carpentersville, Ill.), under the trade name DURAMAC. Resins bearing these family names are believed to be suitable for use as a construction adhesive so long as they are solid at room temperature and are free of volatile solvents.

Although the application of the present invention is not limited to consumer products or articles falling within specific categories, a representative, non-limiting list of such categories includes baby care, feminine protection, incontinence care, paper products, and household care articles. A variety of product forms may fall within each of these categories. Exemplary baby care articles include diapers, wipes, baby bibs, baby change and bed mats. Exemplary feminine protection articles include pads, tampons, interlabial products, and pantyliners. Exemplary incontinence care articles include diapers, pads, and liners. Exemplary paper products include toilet tissues, paper towels, and facial tissues. Exemplary household care articles include sweeper products, and floor cleaning products.

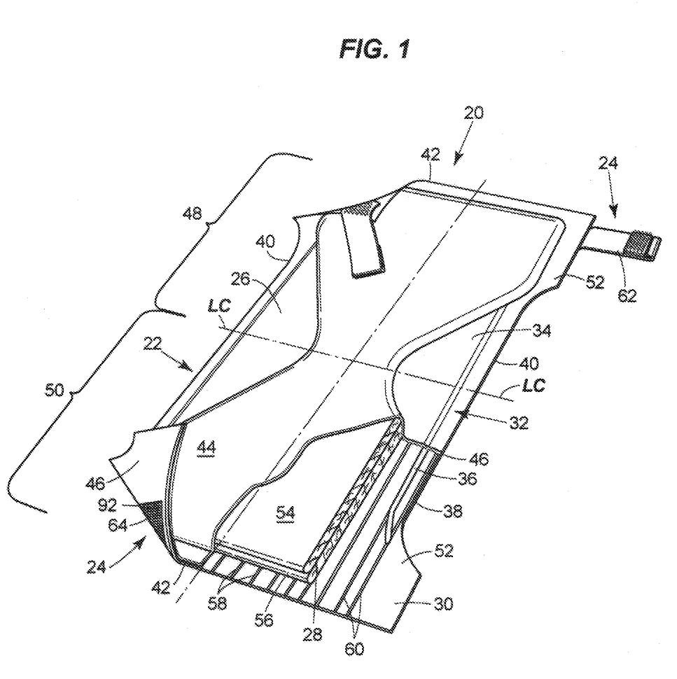

Referring now to the drawings figures, FIG. 1 is a partially cut-away perspective view of a disposable absorbent article (e.g., a diaper) 20 prior to its being placed on the diaper wearer (e.g., an infant) by the diaper user (e.g., a parent). As shown in FIG. 1, the article 20 includes a body portion 22 and a fastening system 24, which is described in more detail below. The body portion 22 includes a liquid pervious topsheet 26, an absorbent core 28, a liquid impervious backsheet 30, and leg cuffs 32 that include a side flap 34. In one embodiment, the leg cuffs 32 are elastically contractible and, therefore, include one or more elastic members 36. The topsheet 26, the absorbent core 28, the backsheet 30, the side flaps 34, and the elastic members 36 may be assembled in a variety of well known disposable diaper configurations, such as, for example, those shown and described in Buell U.S. Pat. No. 3,860,003.

FIG. 1 shows an embodiment of the body portion 22 in which the topsheet 26 and the backsheet 30 are coextensive and have length and width dimensions generally larger than those of the absorbent core 28. The topsheet 26 is superposed on the backsheet 30 thereby forming a periphery 38 of the body portion 22. The periphery 38 defines the outer perimeter or, in other words, the outer extent of the body portion 22. The periphery 38 includes longitudinal edges 40 and end edges 42.

The body portion 22 has an inside surface 44 and an outside surface 46. In general, the outside surface 46 of the article 20 extends from one end edge 42 to the other end edge 42 of the diaper 20 and from one longitudinal edge 40 to the other longitudinal edge 40 of the diaper 20 and is the surface farthest from the wearer during use of the article 20. When a backsheet 30 is used, it typically forms the outside surface 46 of the body portion 22. The inside surface 44 is that surface of the article 20 opposite the outside surface 46 and, in the embodiment shown in FIG. 1, is typically formed by the topsheet 26. In general, the inside surface 44 of the article 20 is that surface coextensive with the outside surface 46, and which is for the greater part in contact with the wearer when the article 20 is worn. The inside surface 44 also is often referred to as the “body-facing” surface of the article 20, while the outside surface 46 is often referred to as the “garment-facing” surface.

The article 20 has first and second end regions 48 and 50, respectively, extending from the end edges 42 of the diaper periphery 38 toward the lateral centerline (denoted “LC” in FIG. 1) of the article 20. Both the first end region 48 and the second end region 50 extend a distance of about one-half of the length of the article 20 such that the end regions comprise each half of the article 20.

Both the first end region 48 and the second end region 50 have panels 52. The panels 52, also referred to herein as ear tabs, are those portions of the first end region 48 and the second end region 50 that overlap with one another when the article 20 is fastened about the waist of the wearer. The extent to which the end regions 48 and 50 overlap and, thus, the extent to which the panels 52 are formed will depend on the overall dimensions and shape of the article 20 and the size of the article wearer.

The absorbent core 28 of the body portion 22 may be any material that is generally compressible, conformable, non-irritating to the wearer's skin, and capable of absorbing and retaining liquids and certain body exudates, such as urine or other fluids and fecal matter, discharged by an incontinent wearer of the article. The absorbent core 28 may be manufactured in a wide variety of sizes and shapes (e.g., rectangular, hourglass, T-shaped, asymmetric, etc.) and from a wide variety of liquid absorbent materials commonly used in disposable diapers and other disposable absorbent articles, such as comminuted wood pulp, generally referred to as airfelt, and comminuted and airlaid wood pulp, commonly referred to as absorbent fluff. Examples of other suitable absorbent materials include creped cellulose wadding, meltblown polymers, chemically-stiffened, modified, or cross-linked cellulosic fibers, tissue, absorbent foams including those prepared from polymerization of a high internal phase emulsion, absorbent sponges, superabsorbent polymers, absorbent gelling materials, or any other known absorbent materials or combination of materials. The total absorbent capacity of the absorbent core 28 should, however, be compatible with the design exudate loading in the intended use of the diaper 20. Further, the size and absorbent capacity of the absorbent core 28 may be varied to accommodate wearers ranging from infants to adults.

While the absorbent core 28 may include a single layer of absorbent material sure as the configuration described in Weisman et al. U.S. Pat. No. 4,610,678, in one embodiment, the absorbent core 28 is a dual-layered absorbent core in a configuration such as is generally described in Weisman et al. U.S. Pat. No. 4,673,402, having an asymmetric-shaped upper layer 54 and a lower layer 56. According to one embodiment, the upper layer 54 acts as a liquid acquisition/distribution layer primarily constructed of hydrophilic fiber material. The lower layer 56 acts as a fluid storage layer containing a mixture of hydrophilic fiber material and particles of an absorbent gelling material (hydrogel material). Both the upper layer 54 and the lower layer 56 include an absorbent layer encased in a tissue layer. The size, shape, configuration, and total absorbent capacity of the upper layer 54 or the lower layer 56 may be varied to accommodate wearer's ranging from infants through adults. Therefore, the dimensions, shape, and configuration of both the upper layer 54 and the lower layer 56 may be varied (e.g., the upper layer 54 or the lower layer 56 may have a varying caliper, a hydrophilic gradient, a rapid acquisition zone or may contain absorbent gelling material).

The absorbent core 28 is superposed on the backsheet 30 and, in one embodiment, is associated thereto by a core attachment means 58, such as those well known in the art, for example, pressure-sensitive adhesives, hot-melt adhesives or other adhesives, ultrasonic bonding; or heat/pressure sealing. Preferably, however, the core attachment means 58 is the adhesive containing the water-stable, oil-modified, nonreactive alkyd resin, as described herein. The absorbent core 28 may be secured to the backsheet 30 by a uniform continuous layer of adhesive, a patterned layer of adhesive, or any array of separate lines or spots of adhesive. The core attachment means 58 can include an open pattern network of filaments of adhesive as is shown in Minetola et al. U.S. Pat. No. 4,573,986.

The backsheet 30 is impervious to liquids and, in one specific embodiment, is manufactured from a thin plastic film, although other flexible liquid impervious materials also may be used. The backsheet 30 prevents the liquids and exudates absorbed and contained in the absorbent core 28 from soiling garments that might contact the article 20, such as bed-sheets and undergarments. In one embodiment, the backsheet 30 is a polyolefin (e.g., polyethylene) film having a thickness of from about 0.012 mm (0.5 mil) to about 0.051 mm (2.0 mils), although other flexible, liquid impervious materials may be used. As used herein, the term “flexible” refers to materials that are compliant and that will readily conform to the general shape and contours of the human body. The backsheet 30 may be embossed and/or matte-finished to provide a more cloth-like appearance. Further, the backsheet 30 may permit vapors to escape from the absorbent core 28 while still preventing liquids and exudates from passing through the backsheet 30. The size of the backsheet 30 generally will be determined by the size of the absorbent core 28 and the exact diaper design selected, for example. In one embodiment, the backsheet 30 has a modified hourglass shape extending beyond the absorbent core a minimum distance of at least about 1.3 cm to about 2.5 cm (about 0.5 to about 1.0 inch) around the entire diaper periphery 38.

The topsheet 26 of the body portion 22 is compliant, soft feeling, non-irritating (to the wearer's skin) planar material. Further, the topsheet 26 is liquid pervious, permitting liquids to readily penetrate through its thickness, and freely pass through it into the absorbent element. Its hydrophobic nature tends to cause its body-facing surface to be dryer and, therefore, protected from the fluids absorbed within the absorbent element. A suitable topsheet 26 may be manufactured from a wide range of materials such as porous foams, reticulated foams, apertured films, natural fibers (e.g., wood or cotton fibers), synthetic fibers (e.g., polyester or polypropylene fibers) or from a combination of natural and synthetic fibers. In one embodiment, the topsheet 26 is made of a hydrophobic material, such as a hydrophobic nonwoven fabric, to isolate the wearer's skin from liquids retained in the absorbent core 28. There are a number of manufacturing techniques that may be used to manufacture the topsheet 26. For example, the topsheet 26 may be woven, nonwoven, spunbonded, carded, hydroformed or the like.

The topsheet 26 and the backsheet 30 are associated together in any suitable manner as is well known in the diaper manufacturing art. As used herein, the term “associated” encompasses configurations whereby the topsheet 26 is directly joined to the backsheet 30 by affixing the topsheet 26 directly to the backsheet 30, and configurations whereby the topsheet 26 is indirectly joined to the backsheet 30 by affixing the topsheet 26 to intermediate members which in turn are affixed to the backsheet 30. In one embodiment, the topsheet 26 and the backsheet 30 are joined directly to each other in the diaper periphery 38 by a flap attachment means 60 such as an adhesive (including an adhesive containing a water-stable, oil-modified, nonreactive alkyd resin as described herein) or any other attachment means as is known in the art. In general, the core attachment means 58 that affixes the absorbent core 28 to the backsheet 30 is the same means as the flap attachment means 60 that affixes the topsheet 26 to the backsheet 30. Thus, for example, a uniform continuous layer of adhesive, a patterned layer of adhesive, an array (e.g., spiral) of separate lines or spots of adhesive, or a network, or adhesive filaments such as shown in U.S. Pat. No. 4,573,986 may be used.

Leg cuffs 32, such as elastically contractible leg cuffs, are positioned adjacent the periphery 38 of the body portion 22, such as along each longitudinal edge 40, so that the leg cuffs 32 tend to draw and hold the article 20 against the legs of the wearer. While the leg cuffs 32 may include any of several means as are well known in the diaper art, one specific embodiment of the leg cuff construction includes a side flap 34 and one or more elastic members 36, as is described in detail in Buell U.S. Pat. No. 3,860,003. Additionally, a method and apparatus suitable for manufacturing a disposable diaper having elastically contractible leg cuffs are described in Buell U.S. Pat. No. 4,081,301, Lawson U.S. Pat. No. 4,695,278, Dragoo U.S. Pat. No. 4,795,454, and Buell U.S. Pat. No. 4,900,317. In another embodiment, the elastically contractible leg cuff 32 includes a side flap 34 and an elastic member 36 including an elastic thread.

The article (diaper) 20 is provided with a fastening system (one embodiment of which is generally designated 24) for forming a side closure. The fastening system generally includes a fastener 62 and a landing members 64 for receiving the fastener. The diaper 20 is fitted to the wearer and the first end region 48 and the second end region 50 are maintained in an overlapping configuration by the fastening system 24 when the diaper 20 is worn. Embodiments of the fastener 62 are intended to engage the landing member 64 so as to provide a secure side closure for the diaper 20 when worm by the diaper wearer.

The fastener 62 is fixed to the article 20 and is positioned on the outside surface 46 of the body portion 22 in the panels (ear tabs) 52 in the first end region 48, one adjacent each longitudinal edge 40, so as to engage the landing member 64 positioned in the second end region 50. The fastener 62 is fixed to the body portion 22 and, in one embodiment, covers an area about 1 inch wide (i.e., generally perpendicular to longitudinal centerline (designated “LC” in FIG. 1)) by about 2.5 inches long (i.e., generally parallel to the longitudinal centerline) at the panels 52 of the body portion 22. The fastener may generally be considered either as being out-board of the article periphery when the fastener is in a closed position or in-board of the article periphery when in the closed position.

Fastener 62 generally is affixed to the body portion 22 by fastener attachment means capable of providing an adequate bond, such as, for example, heat bonds, pressure bonds, ultrasonic bonds, dynamic mechanical bonds, or any other suitable attachment means or combinations of these attachment means as are known to those of ordinary skill in the art. The fastener attachment means may include any of those adhesives capable of providing an adequate bond with other portions of the diaper. Furthermore, the fastener attachment means can be the adhesive containing the water-stable, oil-modified, nonreactive alkyd resin, as described herein.

Other non-limiting examples of absorbent articles according to the present invention are sanitary napkins designed to receive and contain vaginal discharges, such as menses. Disposable sanitary napkins are designed to be held adjacent to the human body through the agency of a garment, such as an undergarment or a panty or by a specialty designed belt. Examples of the kinds of sanitary napkins to which the present invention is readily adapted are shown in U.S. Pat. Nos. 4,687,478 and 4,589,876.

Generally, sanitary napkins include a liquid impervious backsheet, a liquid pervious topsheet, and an absorbent core placed between the backsheet and the topsheet. The backsheet typically includes a thermoplastic polymer composition. The topsheet may include any of the topsheet materials discussed with respect to diapers. Similarly, the absorbent core may include any of the absorbent core materials discussed with respect to diapers. It will be apparent that construction adhesive described herein can be used to adhere various components of the sanitary napkin to one another. On the other hand it will be understood the present invention is not limited to any specific sanitary napkin configuration or structure.

Sanitary paper tissue products are commercially offered in formats tailored for a variety of uses including facial tissues and napkins, toilet tissues and absorbent towels (e.g., paper towels). These products often include two or more plies of tissue, the material for which is selected from conventional tissue or special kinds of tissues, such as so-called “through-air-dried tissue” or “differential-density-tissue.” The latter is often made via a patterned paper making belt and through-air-drying technology. Generally, however, these products can be made by any process known in the art, including, for example, a conventional papermaking process and a through-air-drying papermaking process. These products employ conventional adhesives that can now be replaced by an adhesive containing a water-stable, oil-modified, nonreactive alkyd resin, as described herein.

A dishwashing wipe generally includes as least two substrates, namely a cleaning substrate and a scrubbing substrate. More specifically, a dishwashing wipe includes a first or top surface that includes a cleaning substrate, and a second or bottom surface that includes a scrubbing substrate. The cleaning substrate generally includes a nonwoven fabric and the scrubbing substrate generally includes a scrim. These wipes often additionally contain one or more layers of batting, and a dishwashing composition in the form of a plurality of strips of paste between the cleaning substrate and the layers of batting. The component parts of the wipe can be held together by the adhesive composition described herein. The cleaning substrate provides a softer surface when compared with the comparatively more abrasive scrubbing substrate. As used herein, “scrim” refers to any durable material that provides texture to the surface-contacting side of the wipe's scrubbing substrate, and also has a sufficient degree of openness to allow movement of fluid to an absorbent layer of the wipe. Suitable scrim materials include those that have a continuous, open structure, such as synthetic and wire mesh screens. The open areas of these scrim materials can be readily controlled by varying the number of interconnected strands that make up comprise the scrim, by controlling the thickness of those interconnected strands, etc. These products employ conventional adhesives that can now be replaced by an adhesive containing a water-stable, oil-modified, nonreactive alkyd resin, as described herein.

### EXAMPLES

The following examples are provided to illustrate the invention, but are not intended to limit the scope thereof. The experiments described in Example 1 demonstrate that an alkyd resin remains fluid over a broader temperature range than does a conventional hot-melt adhesive, which is desirable because it makes possible opportunities to adhere temperature-sensitive substrates. The experiments described in Example 2 demonstrate that the tested alkyds have a color profile acceptable for the uses in which an adhesive is contemplated and acceptable even in instances where the adhesive is not likely to be visible through the adherends. The experiments described in Example 3 demonstrate the water stability of an alkyd resin, and specifically that the tested resin remained dimensionally unchanged and within target performance criteria for applications in which an adhesive is herein contemplated, and that laminates adhered with the subject alkyd resin-containing adhesive experienced less than a 20% bond strength loss after exposure to ambient-temperature water for at least 16 hours. The experiments described in Example 4 demonstrate the process feasibility of a short oil, non-drying alkyd resin in the context of processing conditions pertinent to the manufacture of a disposable absorbent article. The experiments described in Example 5 demonstrate that the viscosity of an alkyd resin adhesive remains sufficiently constant in the context of what is expected and desired of a construction adhesive employed in the manufacture of a disposable absorbent article.

### Example 1

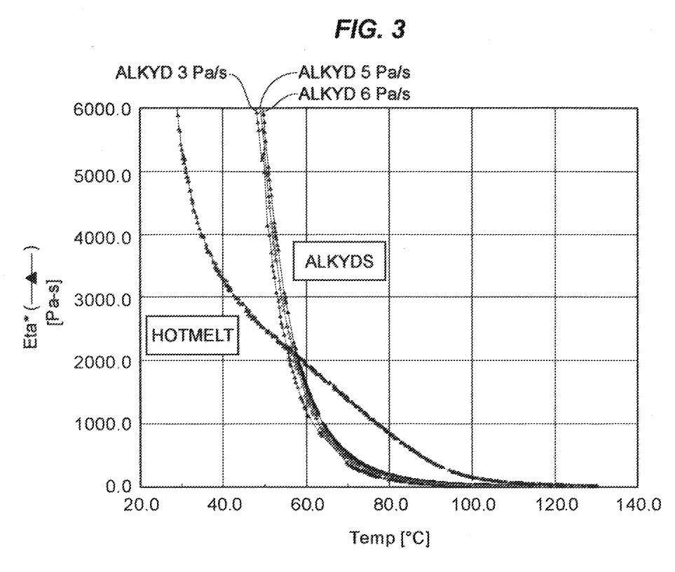

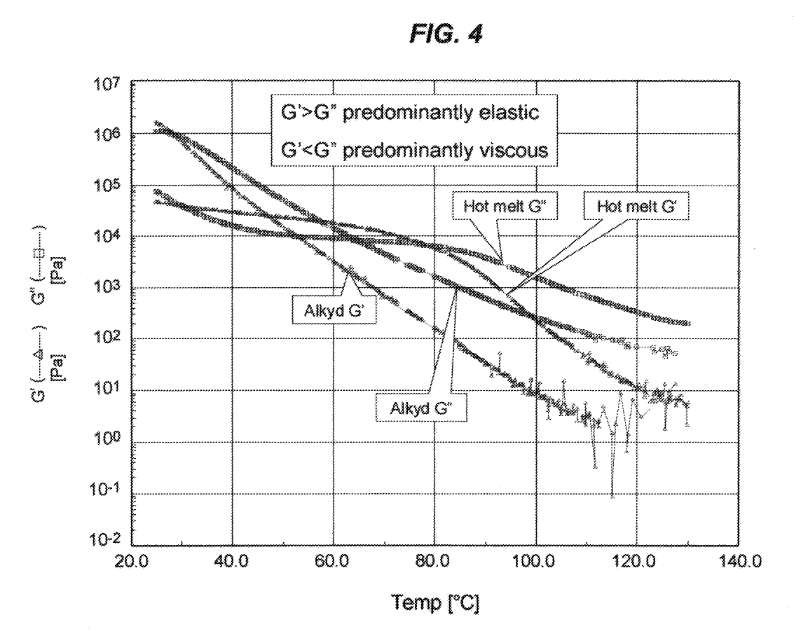

In this example, the dynamic viscosity (η), the storage modulus (G′), and the loss modulus (G″) of different grades of the same alkyd resin are compared to each other and to a conventional hot-melt adhesive typically used to construct disposable absorbent articles. The hot-melt adhesive used in this and other examples herein is commercially available from the National Starch & Chemical Company (Bridgewater, N.J.), under the product name DM 526 (and may now be commercially available from Henkel Adhesive Technology Company (Bridgewater, N.J.), under the product name DISPOMELT® 526). The dynamic viscosity (η), storage modulus (G′), and loss modulus (G″) of the materials were measured over a temperature range of about 20° C. to about 130° C., and are reported in FIGS. 3 and 4.

The alkyd resin was prepared from a short oil, non-drying alkyd resin commercially available from Reichhold Chemicals Inc. (Durham, N.C.), under the trade name BECKOSOL® 12-035. The obtained resin was manufactured with coconut oil, had a viscosity of 1.9 Pascals/second (Pa/s), and contained 2% to 3% solvent (xylene). As obtained, this specific resin is referred to herein as the “fresh alkyd.” The fresh alkyd was heated to a temperature of 150° C. to 200° C. for about 14 minutes to about 23 minutes to strip the fresh alkyd of its solvent and thereby provide what is referred to herein as the “cooked alkyd.” Three grades of the cooked alkyd were prepared by the foregoing heating—one having a viscosity of 3 Pa/s, a second having a viscosity of 5 Pa/s, and a third having a viscosity of 6 Pa/s. These grades were achieved based on the duration of the aforementioned heating, wherein the longer heating times imparted the cooked alkyd with a higher viscosity (due to the increase in alkyd molecular weight). Rheological assessments (η, G′, and G″) were conducted using a rheometer Model SR5 from Rheometrics Scientific (Piscataway, N.J.). The temperature ramp is 130° C. to 25° C., with steps of 2° C. per minute, and the frequency was 10 rad/sec.

FIG. 3 graphically depicts the dynamic viscosity of the four materials over a temperature range of about 20° C. to about 130° C. That depiction demonstrates that the various grades of the cooked alkyd have nearly identical dynamic viscosity (η), relative to one another, over the measured temperature range. The nearly identical dynamic viscosity is indicative of stability. The depiction further demonstrates that the dynamic viscosity of the alkyd is unlikely to be influenced by further heating/cooking in the event a higher molecular weight alkyd is desired.

FIG. 4 graphically depicts the elastic or storage modulus (G′) and the viscous or loss modulus (G″) over a temperature range of about 20° C. to about 130° C. for the conventional hot-melt adhesive (referred to in the figure as “Hot melt”) and one of the grades of the cooked alkyd (specifically the 5 Pa/s grade, referred to in the figure as “Alkyd”). That depiction demonstrates that the conventional hot-melt adhesive exhibits viscous-elastic behavior in the temperature range of about 30° C. to about 70° C., and exhibits viscous behavior between a different temperature range of about 80° C. to about 120° C. In contrast, the cooked alkyd does not exhibit viscous elastic behavior in the temperature range of about 30° C. to about 130° C. Furthermore, the cooked alkyd exhibits viscous behavior over a temperature range of about 30° C. to about 130° C.—a much broader temperature range than the one in which the conventional hot-melt adhesive exhibited viscous behavior.

The broad temperature range over which the cooked alkyd remains fluid (relative to the narrow range in which conventional hot-melt adhesives remain fluid) is important because it now provides artisans with an opportunity to apply the adhesive to a broader class of adherends, specifically to adherends that have low melting points. More specifically, conventional hot-melt adhesives are not typically used to adhere together adherends that are susceptible to damage by the temperature at which the adhesive would need to be applied (in its low viscosity state). For example, a conventional hot-melt adhesive would need to be heated to a temperature of about 120° C. to attain a sufficiently low viscosity that it can be applied as a liquid to an adherend. If, however, the adherend is polyethylene, which has a melting point of about 120° C., the adherend may itself melt when contacted by the liquefied, conventional hot-melt adhesive. In desirable contrast, the alkyd has low viscosity at lower temperatures and, therefore, it can be applied with success to adhere polyethylene substrates without the potential of damaging such substrates. Consequently, a broader class of adherends may now be adhered.

### Example 2

In this example, the Gardner-Color grade of different grades of the same alkyd resin are compared to each other and to a conventional hot-melt adhesive typically used to construct disposable absorbent articles. Two grades of alkyd resin described in Example 1 (specifically the 5 Pa/s and the 6 Pa/s grades) and the hot-melt adhesive described in Example 1 were used in this example.

The Gardner-Color is the color of transparent liquids by comparison with standards of specified colors, Gardner-Delta Comparator (CG-6750). The color of the sample is the number of the standard most closely matching the sample. The higher number indicates the darker color of the sample (ASTM D 1544). In the applications in which an adhesive is contemplated, a color grade of “3” or lower is acceptable. Color grades in excess of “3” are acceptable in instances where the adhesive is not likely to be visible and not likely to be visible through the adherends.

The test protocol described at ASTM Standard D 1544, “Test Method for Color of Transparent Liquids (Gardner Color Scale),” ASTM International, West Conshohocken, Pa., 2004, DOI: 10.1520/D1544-04, www.astm.org, was used to carry out the experiments, with Lovibond Gardner Comparator 3000 AF 228 for color grade assessment.

Based on the foregoing test protocol, the conventional hot-melt adhesive exhibited a color grade of “1,” where as the fresh alkyd exhibited a color grade of “2,” and the cooked alkyds (both the 5 Pa/s and the 6 Pa/s) after 72 hours in an open container exhibited a color grade of “3.” The results demonstrate that the fresh and cooked alkyds have a color profile acceptable for the uses in which an adhesive is contemplated. Furthermore, the results demonstrate that the fresh and cooked alkyds likely will be acceptable even in instances where the adhesive is not likely to be visible through the adherends.

### Example 3

In this example, the water stability/adhesion performance of an alkyd resin are compared to that of a conventional hot-melt adhesive typically used to construct disposable absorbent articles. The same hot-melt adhesive described in Example 1 and the 5 Pa/s grade of alkyd resin described in Example 1 were used in this example.

The performance of an adhesive formulation can be tested by measuring its bond strength before and after exposure to ambient-temperature (e.g., 23° C.) water for at least 16 hours. For example, the performance of an adhesive formulation can be tested by measuring the bond strength of two nonwoven layers adhered together by the subject adhesive formulation to form a laminate, before and after exposure to ambient-temperature water for at least 16 hours.

Three test laminates were prepared. A first laminate was prepared by extruding three stripes of the conventional hot-melt adhesive via a slot coater between two layers of nonwoven material, one of which was a nonwoven SMS 10 grams per square meter (gsm), hydrophilic material, whereas the other layer was a nonwoven SMS 11 gsm, hydrophobic material. The adhesive was extruded from the slot coater at a temperature of 150° C., and applied at a coater speed of 400 meters/minute. The distance between the application site on the nonwoven material and the slot coater was 500 mm, the stripes were 1 mm wide, and the basis weight of the stripes was 27 gsm.

A second laminate was prepared in the same manner as the first laminate, except that it employed the 5 Pa/s grade alkyd (instead of the conventional hot-melt adhesive), which was extruded from the slot coater at a temperature of 135° C.

A third laminate was repared in the same manner as the second laminate, except that the stripes of the 5 Pa/s grade alkyd had a weight basis of 34 gsm.

The resulting laminates were stored at 23° C. for 24 hours. Samples of each laminate were cut 1-inch side comprising the three adhesive stripes. The bond strength (in units of grams (g)) between the two nonwovens in cross directions was tested. The tensile tester was a Zwick/Roell Z1.0/TH1S (Atlanta, Ga.), with a crosshead speed of 600 mm/min. The resulting force value (g) for each tested sample was taken as the average of the peaks given by the three adhesive stripes. The bond strength between the two nonwovens in cross direction was tested again following exposure of each sample to ambient-temperature water for 16 hours. This test protocol is referred to herein as the “Bond Strength” test.

The first laminate (employing the conventional hot-melt adhesive) had a bond strength before water exposure of 218 g, and 213 g following exposure of the laminate sample to ambient-temperature water for 16 hours. Accordingly, the first laminate experienced a 2.3% loss in bond strength.

The second laminate (employing the 5 Pa/s alkyd resin, at 27 gsm) had a bond strength before water exposure of 101 g, and 128 g following exposure of the laminate sample to ambient-temperature water for 16 hours. Accordingly, the second laminate experienced a 23.7% increase in bond strength.

The third laminate (employing the 5 Pa/s alkyd resin, at 27 gsm) had a bond strength before water exposure of 199 g, and 217 g following exposure of the laminate sample to ambient-temperature water for 16 hours. Accordingly, the third laminate experienced a 9.0% increase in bond strength.

Generally, water stability is evident if the laminate (and accordingly adherends) adhered by the subject adhesive experiences a bond strength loss of less than 20% after the laminate has been exposed to water for at least 16 hours. Preferably the loss is less than 15%, more preferably, less than 10%, and even more preferably less than 5%.

The water stability of the materials (and, generally, of adhesive compositions and articles comprising the compositions) is further measured by charging 1000 milliliters (ml) of tap water (23° C.) to a clean glass beaker. Six one-inch samples are placed in the water for 16 hours. The particular size of the test piece can vary depending on the type of article and method of construction; but, in general, the smallest dimension of the test piece should be smaller than 5 millimeters (mm). Thereafter the test pieces are removed and dried. The pieces then are measured again and, if the dimensions of the samples change along any length by less than 20%, then the samples and, consequently, the thermoplastic composition or article from which the test piece is made, is said to display water stability. Preferably, the dimensions of the samples change along any length by less than 10%, and even more preferably less than 5%. Based on the foregoing test, the tested laminates remained dimensionally unchanged and within target performance criteria for applications in which an adhesive is herein contemplated.

### Example 4

In this example, the process feasibility of a short oil alkyd resin is demonstrated in an experiment performed to simulate a manufacture of a disposable absorbent article, namely a diaper.

The following materials were used in carrying out this experiment: (1) a 10 grams per square meter (gsm) nonwoven SMS-type hydrophilic (Cover layer); (2) a 11 gsm nonwoven SMS-type hydrophobic (Dustin layer); (3) a short oil, non-drying alkyd resin commercially available from Reichhold Chemicals Inc. (Durham, N.C.), under the trade name BECKOSOL® 12-035, which was further treated to remove solvent (xylene) present in the commercial product to yield a solvent-free material having a viscosity of 5 Pa/s (cooked alkyd); and, (4) a 14 gsm non-breathable polypropylene film (backsheet).

Generally, the nonwoven cover layer was adhered to the nonwoven Dustin layer by the cooked alkyd through a slot application of the resin. The backsheet was adhered to the nonwoven Dustin layer by the cooked alkyd through a spiral application of the resin. The slot and spiral applications of adhesive are known by those skilled in the art and a general description of these applications has been set forth above.

For the slot application of this experiment, the equipment included a standard hot-melter ITW S05. pump size 2.4 cubic centimeters per revolution (cc/rev); and a Glue Gun Nordson SCSE 250, customized five modules, shim thickness of 0.15 mm. The temperature at which the alkyd was applied with the glue gun was 130° C. The slot application of the alkyd resin was performed directly onto the nonwoven cover layer and then combined with the nonwoven Dustin layer. The pattern application simulated the inner side seal and outer side seal slot. The tested line speed during (chassis) application was about 400 meters/minute (m/min). The amount of alkyd resin applied was 27 gsm to 34 gsm.

For the spiral application of this experiment, the equipment included the same standard hot-melter used in the slot application. The spiral application, however, used a different glue gun—specifically, a Glue Gun Nordson CF 201, Nozzle 0.018WCW. The temperature at which the alkyd was applied with the glue gun was 140° C. to 145° C. The spiral application of the alkyd resin was performed directly onto the polymeric film and then combined with the nonwoven Dustin layer. The tested line speed during (chassis) application was about 400 m/min. The amount of alkyd resin applied was 3.5 gsm to 8 gsm.

The results of the slot and spiral applications demonstrate that the cooked alkyd resin can be processed at industrial line-speeds and that the resin exhibits (initial) bond strength similar to that of a conventional hot-melt adhesive. Images of the spiral pattern demonstrate a well-defined geometry of that pattern.

### Example 5

In this example, the heat (or thermal) stability of different grades of the same alkyd resin are compared to each other. The same grades of alkyd resin described in Example 1 were used in this example, except that the 3 Pa/s grade was not part of this experiment.

The test protocol described at ASTM Standard D 4499-07, “Standard Test Method for Heat Stability of Hot-Melt Adhesives,” ASTM International, West Conshohocken, Pa. 2004, DOI: 10.1520/D4499-07, www.astm.org, was used to carry out the experiments, and is referred to herein as the “Thermal Stability” test. Viscosity was measured as in Example 1.

The test conditions included a 130°° C. aging temperature for an aging period of 72 hours. The table below provides pertinent data from the experiment.

TABLE 1

| | | Cooked | Cooked |
|---|---|---|---|
| | Fresh | Alkyd | Alkyd |
| | Alkyd | (5 Pa/s) | (6 Pa/s) |
| Viscosity | 1.99 | 4.6 | 5.7 |
| @ 0 Hours | | | |
| Viscosity | 3.49 | 6.6 | 8.2 |
| @ 72 hours | | | |
| Viscosity | 42% | 43% | 44% |
| Change | | | |
| Skin? | No | Yes | Yes |

The reported viscosity has units of Pa/s, and the viscosity values reported at 72 hours for the open, cooked alkyds was obtained by measuring the viscosity of the material beneath the formed skin. A viscosity change of 50% or less is acceptable for construction adhesives employed in the manufacture of a disposable absorbent article, such as a diaper, and such change is considered as a “substantially constant viscosity” in that context.

All documents cited in the Detailed Description of the Invention are, in relevant part, incorporated herein by reference; the citation of any document is not to be construed as an admission that it is prior an with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A construction adhesive composition comprising:
(a) at least about 65 wt. % of a water-stable, oil-modified, nonreactive alkyd resin; and,
(b) about 0.01 wt. % to about 35 wt. % of one of (i) a polymer selected from the group consisting of polyhydroxyalkanoates, polyvinyl alcohol, polyethylene, polypropylene, polyethylene terephthalate, maleated polyethylene, maleated polypropylene, polylactic acid, modified polypropylene, nylon, caprolactone, and combinations thereof; and, (ii) a processing aid selected from the group consisting of talc, clay, pulp, flour, walnut shells, cellulose, cotton, jute, raffia, rice chaff, animal bristles, chitin, granular starch, diatomaceous earth, carbon fibers, kenaf, magnesium stearate, fatty acid amides, metal salts of fatty acids, wax acid esters and their soaps, montan wax acids, esters and their soaps, polyolefin waxes, non polar polyolefin waxes, natural and synthetic paraffin waxes, fluoro polymers, silicon, diatomaceous earth, and combinations thereof.

2. The construction adhesive composition of claim 1, wherein the alkyd resin is present in the composition in an amount of at least about 80 wt. %.

3. The construction adhesive composition of claim 1, wherein the alkyd resin is present in the composition in an amount of at least about 90 wt. %.

4. The construction adhesive composition of claim 1, wherein the processing aid is present in an amount of about 0.1 wt. % to about 3 wt. %, based on the total weight of the composition.

5. The construction adhesive composition of claim 1, wherein the alkyd resin comprises a fatty acid or oil selected from the group consisting of capric acid, caproic acid, caprylic acid, lauric acid, lauroleic acid, linoleic acid, linolenic acid, myristic acid, myristoleic acid, oleic acid, palmitic acid, palmitoleic acid, stearic acid, beef tallow, castor oil, coconut oil, coconut seed oil, corn germ oil, cottonseed oil, fish oil, linseed oil, olive oil, oiticica oil, palm kernel oil, palm oil, palm seed oil, peanut oil, rapeseed oil, safflower oil, soybean oil, sperm oil, sunflowerseed oil, tall oil, tung oil, whale oil, and mixtures thereof.

6. The construction adhesive composition of claim 1, wherein the alkyd resin is prepared from phthalic anhydride.

7. The construction adhesive composition of claim 1, wherein the alkyd resin comprises a short oil alkyd.

8. The construction adhesive composition of claim 1, wherein the composition is free of a free plasticizer.

9. The construction adhesive composition of claim 1, wherein the composition is free of a free tackifier.

10. The construction adhesive composition of claim 1, wherein the alkyd resin has a substantially constant viscosity, as measured according to the Thermal Stability test.

11. The construction adhesive composition of claim 1, wherein the composition has a surface energy of at least about 40 dynes/cm.

12. An article comprising:
(a) article components; and,
(b) an adhesive composition applied between and joining at least two of the article components together, the adhesive composition comprising a water-stable, oil-modified, nonreactive alkyd resin.

13. The article of claim 12, wherein the article is a disposable absorbent article and the article components are disposable absorbent article components.

14. The article of claim 13, wherein the disposable absorbent article components joined by the adhesive composition experience less than 20% loss of bond strength after exposure to water for at least 16 hours, as measured according to the Bond Strength test.

15. The article of claim 13, wherein the disposable absorbent article components joined by the adhesive composition experience an increase in bond strength after exposure to water for at least 16 hours, as measured according to the Bond Strength test.

16. The article of claim 13, wherein the disposable absorbent article components comprise a topsheet, a backsheet, and an absorbent core suitable for positioning between the topsheet and backsheet.

17. The article of claim 13, wherein the disposable absorbent article is selected from the group consisting of a diapers, pull-on diapers, pant-type garments, training pants, incontinence briefs, incontinence undergarments, absorbent inserts, diaper holders, and liners, and feminine hygiene garments.

18. The article of claim 13, wherein the disposable absorbent article components comprise at least two plies of a tissue.

19. The article of claim 18, wherein the disposable absorbent article is selected from the group consisting of paper towels, napkins, wipes, and handkerchiefs.

20. The article of claim 12, wherein the article is a packaging article and the article components are packaging article components.

21. A method of forming an adhesive bond between components of an article, the method comprising:
(a) dispensing onto a first article component an adhesive composition comprising a water-stable, oil-modified, nonreactive alkyd resin at a temperature of about 80° C. to about 200° C.; and,
(b) joining the first article component with a second article component to form an adhesive bond as the adhesive cools to room temperature.

22. The method of claim 21, wherein the adhesive bond experiences less than 20% loss of bond strength after exposure to water for at least 16 hours, as measured according to the Bond Strength test.

23. The method of claim 21, wherein the adhesive bond experiences an increase in bond strength after exposure to water for at least 16 hours, as measured according to the Bond Strength test.
